(19) United States
(12) Patent Application Publication
(10) Pub. No.: US 2025/0268435 A1
(43) Pub. Date: Aug. 28, 2025

(54) Smart Toilet Seat Assembly, Load Sensor, and Fixing Arrangement

(71) Applicant: Stichting IMEC Nederland, Wageningen (NL)

(72) Inventors: Eva Wentink, Heeze (NL); Vera Verbiest, Rhenen (NL); David Young, Eindhoven (NL); Remco Van Hardeveld, Veenendaal (NL)

(21) Appl. No.: 19/061,522

(22) Filed: Feb. 24, 2025

(30) Foreign Application Priority Data
Feb. 28, 2024 (EP) 24160182.2

(51) Int. Cl. A47K 13/24; A61B 5/01; A61B 5/024; A61B 5/053

(52) U.S. Cl. CPC A47K 13/24; A61B 5/01; A61B 5/02416; A61B 5/053

(57) ABSTRACT

A smart toilet seat assembly for a toilet bowl for measuring physiological parameters of a user is provided. The smart toilet seat assembly includes a seat including a plurality of physiological sensors. Each of the plurality of physiological sensors is configured to generate at least one physiological parameter measurement data of the user. The smart toilet seat assembly further includes a plurality of load sensors configured to support the seat on the toilet bowl when used by the user and further to generate a weight measurement data of the user. Moreover, the smart toilet seat assembly includes a fixing arrangement configured to attach a lid to the seat and to attach the assembly to the toilet bowl. The fixing arrangement is configured to transfer a weight of the lid to the toilet bowl and further to transfer a weight applied on the seat to the plurality of load sensors.

### CROSS-REFERENCE TO RELATED APPLICATIONS

The present application is a non-provisional patent application claiming priority to European Patent Application No. 24160182.2, filed Feb. 28, 2024, the contents of which are hereby incorporated by reference.

### FIELD OF THE DISCLOSURE

The disclosure relates to sensor integration in smart health toilet seats, such as for measuring health related parameters of a user.

### BACKGROUND

Generally, regularly measuring health parameters may be used for many diagnosed patients as well as for people at risk. A toilet seat may be used as an easy, non-obtrusive way of measuring health parameters. People with a condition, for instance inflammatory bowel disease (IBD), may also benefit from this way of tracking parameters, since the measurements may be used to notify the user of the condition of their health, which may allow them to take appropriate actions. This may prevent worsening of conditions and may allow monitoring of diseases and of health or may allow tracking of medication.

For example, the document US 2019/0231271 A1 discloses a toilet seat for medical analysis of a seated individual. In this regard, the toilet seat comprises force sensors for measuring changes in the apparent weight of the individual, where the seat is secured to the surface via floating hinges. However, the seat cover is connected to the hinges in a conventional manner, which may affect the measurement accuracy of the force sensors.

### SUMMARY

Accordingly, an object of the disclosure is to provide a smart toilet seat assembly, a load sensor, and a fixing arrangement for measuring health related parameters of a user, thereby improving the measurement accuracy of the integrated sensors.

The first independent claim provides the smart toilet seat assembly, the second independent claim provides the load sensor, and the third independent claim provides the fixing arrangement. The dependent claims contain further disclosure.

According to a first aspect of the disclosure, a smart toilet seat assembly for a toilet bowl for measuring physiological parameters of a user is provided. The smart toilet seat assembly includes a seat including a plurality of physiological sensors. Each of the plurality of physiological sensors is configured to generate at least one physiological parameter measurement data of the user.

The smart toilet seat assembly further includes a plurality of load sensors configured to support the seat on the toilet bowl when used by the user and further to collectively generate a weight measurement data of the user. Moreover, the smart toilet seat assembly includes a fixing arrangement configured to attach a lid to the seat and further to attach the smart toilet seat assembly to the toilet bowl.

In this regard, the fixing arrangement is configured to transfer a weight of the lid (e.g., exclusively) to the toilet bowl and further to transfer a weight applied on the seat (e.g., exclusively) to the plurality of load sensors.

In an example embodiment, the sensor integration in the toilet seat may provide an easy and a non-obtrusive way of measuring health parameters. Furthermore, by transferring the weight of the lid (e.g., directly) to the toilet bowl and by transferring the weight applied on the toilet seat (e.g., exclusively) to the load sensors, a high measurement accuracy of the sensors, such as the load sensors, may be provided.

In an example embodiment, the plurality of physiological sensors are arranged on a top surface of the seat being in physical contact with the user. Additionally or alternatively, at least one sensor of the plurality of physiological sensors is configured to be in contact with the user, such as in physical contact with the user.

In an example embodiment, the plurality of load sensors are arranged on a bottom surface of the seat being in contact with the toilet bowl.

For example, the sensors that use (e.g., require) physical contact with the user may be integrated in the top surface of the seat and the load sensors may be integrated in the bottom surface of the seat to provide an accurate readout of the weight applied on the seat.

In an example embodiment, the plurality of physiological sensors include at least one electrocardiogram (ECG) sensor, at least one bio-impedance (BioZ) sensor, at least one photoplethysmography (PPG) sensor, and at least one temperature sensor.

In this regard, the at least one temperature sensor may not be in direct and/or physical contact with the user. For instance, the at least one temperature sensor may be arranged on the seat such as in a close proximity, e.g., about 30 cm or less, in relation to the user.

Additionally or alternatively, the at least one physiological parameter measurement data includes an electrocardiogram measurement data or a bio-impedance measurement data or a photoplethysmography measurement data or a temperature measurement data of the user.

For instance, the sensors may provide physiological parameter measurement data, which may be processed to extract (e.g., important) health related features such as heart rate, heart rate variability, blood pressure changes including the pulse transit time, pulse arrival time, and pre-ejection period, absolute weight, weight change over time, urine or stool condition, weight loss per toilet session including urine flow rate, the amount of urine, and the amount of stool, bowel movement or pressure, number of toilet sessions, respiration rate, type of respiration, core temperature, position on the seat, oxygen saturation, body composition, inflammation, and/or the like.

In an example embodiment, the seat includes a switching arrangement configured to switch on or switch off the plurality of physiological sensors and/or the plurality of load sensors based on a presence or an absence of the user, respectively.

For instance, the switching arrangement may include a capacitive touch sensor that may detect the presence of the user, such as when the user is sitting on the toilet seat, and may initiate the sensors for sensing and measuring upon detecting the user. In an example embodiment, the switching arrangement may facilitate a power saving scheme in order not to consume too much power (e.g., reduce or minimize power consumption) when the toilet seat is not in use.

In an example embodiment, the lid includes a receiving unit configured to receive the at least one physiological parameter measurement data from the plurality of physiological sensors when arranged on the top surface of the seat and/or the weight measurement data from the plurality of load sensors when arranged on the bottom surface of the seat.

For example, the lid (e.g., the receiving unit of the lid) may be connected to the seat (e.g., to the sensors integrated in the seat) through a wired data link or a wireless data link, and the lid may receive the measurement data from the sensors through the wired data link or wirelessly.

In an example embodiment, the lid includes a processing unit configured to process the at least one physiological parameter measurement data and/or the weight measurement data and further to extract one or more physiological parameters of the user from the at least one physiological parameter measurement data and/or the weight measurement data. For example, in-situ measurement data processing and feedback processing may be provided.

In an example embodiment, the lid includes a display unit configured to display a notification corresponding to the extracted one or more physiological parameters of the user. Thus, user experience may be improved.

In an example embodiment, the lid includes a transmitting unit configured to transmit the one or more physiological parameters of the user to a remote device and/or to a cloud storage (e.g., wirelessly). For example, ex-situ feedback processing, such as for the assessment of health professionals, may be provided.

In an example embodiment, the seat includes a supply unit configured to provide an electrical supply to the plurality of physiological sensors and/or the plurality of load sensors. Thus, the proper operation of the sensors integrated in the toilet seat may be provided.

In an example embodiment, the lid includes a charging unit operably coupled to the supply unit of the seat, the charging unit being configured to transmit an electrical power to the supply unit in order to charge or recharge the supply unit. In an example embodiment, in-situ charging or recharging of the toilet seat in order to provide the operation of the sensors integrated in the toilet seat may be provided.

According to a second aspect of the disclosure, a load sensor is provided for generating a weight measurement data in a smart toilet seat assembly according to the first aspect of the disclosure. The load sensor includes a holding segment configured to attach the load sensor to a seat of the smart toilet seat assembly, a foot segment configured to be in contact with a toilet bowl when used by a user, and a sensor body including a load cell. The sensor body may be arranged between the holding segment and the foot segment.

In this regard, the foot segment includes a crest configured to protrude towards the holding segment through the sensor body. Furthermore, the holding segment includes a magnet configured to align the crest of the foot segment through the sensor body and further to attach the foot segment with the holding segment.

In an example embodiment, the load cell is a strain gauge load cell.

For example, the weight applied on the toilet seat may be measured with a high accuracy and in a cost-effective manner. Furthermore, the magnetic attachment between the holding segment and the foot segment may provide a precise alignment of the segments when the sensor is assembled in the toilet seat. The magnetic attachment may additionally provide cleanability and/or ease of replacing parts.

In an example embodiment, a readout circuit may be integrated in the load sensor for reading out the sensor signal. In an example embodiment, by integrating the readout circuit in the sensor enclosure, sensor data degradation due to path loss may be reduced.

According to a third aspect of the disclosure, a fixing arrangement is provided for attaching a smart toilet seat assembly according to the first aspect of the disclosure to a toilet bowl. In this regard, the fixing arrangement includes at least one hinge.

The at least one hinge includes a hinge body configured to attach the at least one hinge to the toilet bowl. The at least one hinge further includes a first bracket directly coupled to the hinge body, wherein the first bracket is configured to attach a lid of the smart toilet seat assembly to the hinge body in order to transfer a weight of the lid (e.g., exclusively) to the toilet bowl and further to rotate the lid with respect to the hinge body.

Moreover, the at least one hinge includes a second bracket coupled to the hinge body via a coupler. The second bracket is configured to attach a seat of the smart toilet seat assembly to the hinge body via the coupler and further is configured to rotate the seat with respect to the hinge body. In this regard, the coupler is configured to move the seat along a vertical axis based on a weight applied on the seat.

In an example embodiment, by transferring the weight of the lid (e.g., directly) to the toilet bowl and by transferring the weight applied on the toilet seat (e.g., exclusively) to the load sensors, a high measurement accuracy of the sensors, such as of the load sensors, may be provided.

In an example embodiment, the coupler includes a limiting member configured to control the movement of the seat along the vertical axis.

In an example embodiment, the first bracket includes a first damping mechanism configured to control the rotational movement of the lid with respect to the hinge body. Additionally or alternatively, the second bracket includes a second damping mechanism configured to control the rotational movement of the seat with respect to the hinge body.

For example, a softer, slower, and quieter movement of the lid and/or the seat may be facilitated, which may increase the comfort and ease of use.

### BRIEF DESCRIPTION OF THE FIGURES

The above, as well as additional, features will be better understood through the following illustrative and non-limiting detailed description of example embodiments, with reference to the appended drawings.

In the drawings:

FIG. 1A shows a top view of an example embodiment of the smart toilet seat assembly according to an aspect of the disclosure;

FIG. 1B shows a bottom view of the smart toilet seat assembly of FIG. 1A;

FIG. 2A shows a top view of the lid of the smart toilet seat assembly according to an aspect of the disclosure;

FIG. 2B shows a bottom view of the lid of the smart toilet seat assembly of FIG. 2A;

FIG. 3 shows an example operation of the smart toilet seat assembly according to an aspect of the disclosure;

FIG. 4 shows an example charging scheme of the smart toilet seat assembly according to an aspect of the disclosure;

FIG. 5 shows an example embodiment of the hinge according to an aspect of the disclosure;

FIG. 6A shows a front view of the hinge of FIG. 5;

FIG. 6B shows a back view of the hinge of FIG. 5;

FIG. 7A shows an example embodiment of the load sensor according to an aspect of the disclosure;

FIG. 7B shows an exploded view of the load sensor of FIG. 7A;

FIG. 8A shows a front view of the load sensor of FIG. 7A; and

FIG. 8B shows an inside bottom view of a portion of the load sensor of FIG. 7A.

The figures are schematic, not necessarily to scale, and generally show parts which elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and may not be construed as limited to the embodiments set forth herein; rather, these embodiments are provided by way of example. Furthermore, like numbers refer to the same or similar elements or components throughout.

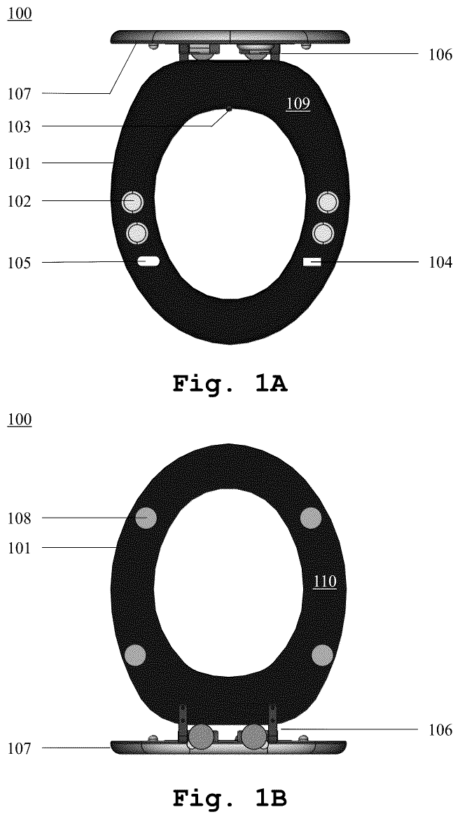

In FIG. 1A and FIG. 1B, an example embodiment of the smart toilet seat assembly 100 is illustrated. FIG. 1A shows a top view of the smart toilet seat assembly 100. The smart toilet seat assembly 100 may include a seat 101 and a lid 107. The seat 101 may include a number of ECG and/or BioZ electrodes 102 on a top surface 109 of the seat 101, such as four ECG and/or BioZ electrodes, for measuring ECG and BioZ of the user of the seat 101 while being in physical contact with the user.

For example, in order to measure ECG and/or BioZ of the user, two source electrodes may be provided to excite from two different measurement locations on the user, and two receiver electrodes may be provided to respectively measure response from the two different measurement locations to realize a tetrapolar or four-electrode measurement technique, such as to accurately generate the ECG measurement data and/or the BioZ measurement data.

For instance, the ECG and/or BioZ electrodes 102 may be operated with multiple frequencies as well as with a single frequency. For instance, a frequency division may be implemented for parallel acquisition of the ECG data (e.g., low frequency) and the BioZ data (e.g., high frequency).

The seat 101 may further include a PPG sensor 104 on the top surface 109 of the seat 101. The PPG sensor 104 may provide measurement data related to the heart rate, oxygen saturation, blood pressure variables, and the like while being in physical contact with the user. The PPG sensor 104 may be arranged on the top surface 109 of the seat 101 to be in physical contact with the leg of the user.

For example, the PPG sensor 104 may include different colors of light-emitting diodes (LEDs). For instance, the PPG sensor 104 may be a PPG module or may include separate LED and/or photodiodes (PDs). Moreover, the PPG sensor 104 may include or be a singular PPG sensor or an array of PPG sensors.

For example, the seat 101 may include an IR temperature sensor 103 that may measure the core temperature of the user. In this regard, the IR temperature sensor 103 may be arranged on the seat 101 such that the IR temperature sensor 103 may perform rectal temperature measurement of the user while using the seat 101, thereby improving the quality and the reliability of the core temperature measurement. In this regard, the IR temperature sensor 103 may include a singular IR sensor or an array of IR sensors.

For example, the seat 101 may include a capacitive touch sensor 105 arranged on the top surface 109 of the seat 101. The capacitive touch sensor 105 may detect when the user is sitting on the seat 101, and may immediately start (e.g., all) sensors for commencing the measurements. In case the presence of the user may not be detected by the capacitive touch sensor 105, e.g., the user is in standing position and/or not using the seat, the capacitive touch sensor 105 may switch off the sensors (e.g., idle state).

For example, the lid 107 may be attached to the seat 101 via a fixing arrangement 106 including two hinges. The fixing arrangement 106 may also attach the smart toilet seat assembly 100 to a toilet bowl (not shown).

FIG. 1B shows a bottom view of the smart toilet seat assembly 100. It may be seen that the seat 101 may include a number of load sensors 108, such as four load sensors, arranged on a bottom surface 110 of the seat 101, such as between the seat 101 and the toilet bowl. The load sensors 108 may collectively measure the weight applied on the seat 101, e.g., the weight of the user sitting on the seat 101.

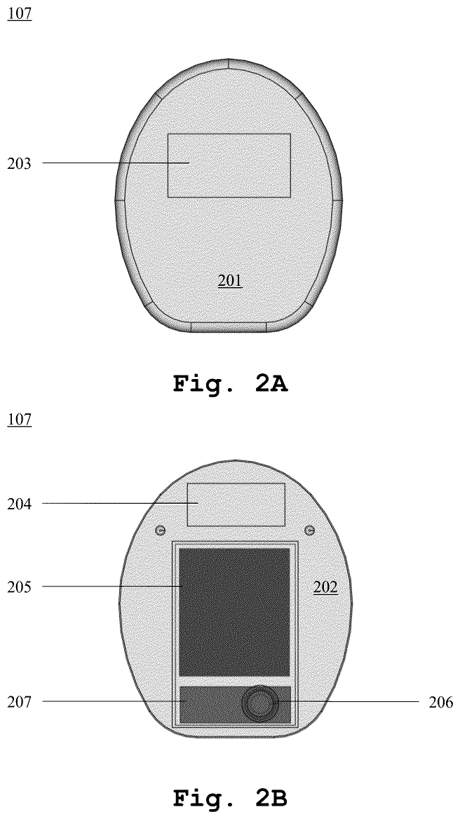

In FIG. 2A and FIG. 2B, an example embodiment of the lid 107 is illustrated is illustrated. FIG. 2A shows a top view of the lid 107. For example, the lid 107 may include a first display 203 arranged on a top surface 201 of the lid 107. The first display 203 may display one or more notifications regarding one or more health related statuses of the user.

FIG. 2B shows a bottom view of the lid 107. For example, the lid 107 may include a second display 204 arranged on a bottom surface 202 of the lid 107. The second display 204 may display one or more notifications regarding one or more health related statuses of the user.

The lid 107 may further include a charging unit 205, a processing unit 207, and a wireless antenna 206 arranged on the bottom surface 202 of the lid 107. The charging unit 205 may charge or recharge the seat 101 via the wireless antenna 206, while the processing unit 207 may receive the measurement data from the seat 101 and may process the measurement data to derive one or more health related parameters.

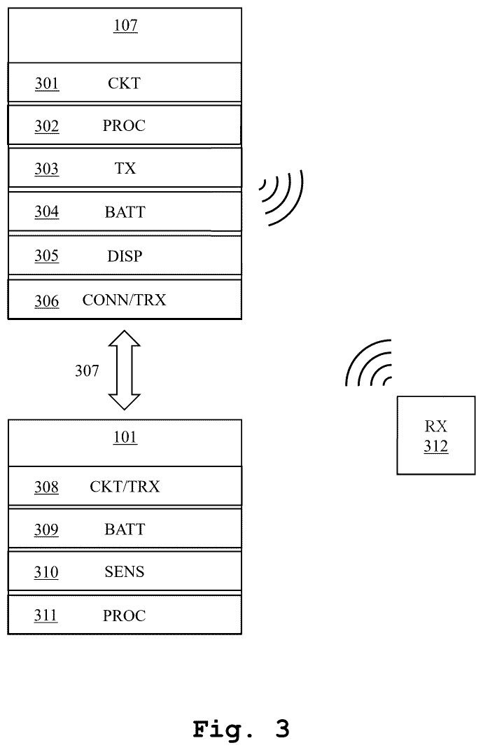

In FIG. 3, an example operation of the smart toilet seat assembly 100 is illustrated. FIG. 3 shows a block representation of the components of the smart toilet seat assembly 100.

For example, the seat 101 may include a transceiver block 308, a battery 309, a sensor block 310, and a processing block 311.

For example, the sensor block 310 may include the ECG and/or BioZ sensors 102, the IR temperature sensor 103, the PPG sensor 104, the capacitive touch sensor 105, and the load sensors 108. The battery 309 may be a rechargeable battery, which may provide electrical power to the sensor block 310.

For example, the processing block 311 may include a processor, which may regulate the timing operation of the sensor block 310, e.g., to regulate the timing operation of the ECG and/or BioZ sensors 102, the IR temperature sensor 103, the PPG sensor 104, the capacitive touch sensor 105, and the load sensors 108.

For example, the transceiver block 308 may transmit the measurement data, either wired or wirelessly, from the sensor block 310, such as from the ECG and/or BioZ sensors 102, the IR temperature sensor 103, the PPG sensor 104, and the load sensors 108, to the lid 107. In addition, the transceiver block 308 may receive electrical signals, either wired or wirelessly, from the lid 107 to charge or recharge the battery 309. Additionally, the electrical signals communicated between the lid 107 and the transceiver block 308 may include control signals for turning on/off the processing block 311, switching on/off (e.g., certain) signals and/or sensors, and the like.

Additionally or alternatively, the seat 101 may include a charging interface, which may be connected to a power socket to allow direct charging or recharging of the battery 309.

Additionally or alternatively, the processing block 311 may process the sensor data and may derive one or more health related parameters from the sensor data. In this regard, the transceiver block 308 may transmit the raw sensor data and/or the processed one or more health related parameters to a remote device 312, such as wirelessly.

The remote device 312 may be a wireless receiving device, such as one or more cell phones, PDAs, or may be a signal analysis device, such as one or more VNAs, personal computers. Additionally or alternatively, the remote device 312 may be a remote storage device, such as a network storage or a cloud storage.

For example, the lid 107 may include a charging circuit block 301, such as a wireless charging circuit, a processing block 302, a transmitter block 303, such as a wireless transmitter, a battery pack 304, a display block 305, and a connection block 306.

For instance, the charging circuit block 301 may include a wireless charging antenna, which may be covered with a flexible material, such as silicon, and be pushed out to make proper contact with the seat 101 when the lid 107 is closed. In this regard, the charging circuit block 301, by means of the battery pack 304 and the wireless charging antenna, may provide wireless charging to the seat 101, such as to the battery 309 of the seat 101.

For example, the lid 107 may be independently removed or detached from the smart toilet seat assembly 100. As such, the lid 107 may be used as a wireless power bank for the seat 101, and may be detached and the battery pack 304 may be recharged. In this regard, the lid 107 may include a charging interface, e.g., an USB interface, which may be connected to a power socket for charging or recharging of the battery pack 304.

For example, the connection block 306 may include a wired or wireless link 307, and may receive the measurement data from the ECG and/or BioZ sensors 102, the IR temperature sensor 103, the PPG sensor 104, and the load sensors 108 via the link 307. In this regard, the processing block 302 may include a processor, which may process the sensor data and may derive one or more health related parameters from the measurement data.

For example, the processing block 302 may display one or more health related statuses to the user via the display block 305. The display block 305 may include one or more displays, e.g., as described in FIG. 2A and FIG. 2B.

For example, the transmitter block 303 may transmit the raw measurement data and/or the processed one or more health related parameters to the remote device 312, such as wirelessly.

Although not shown, the seat 101 and/or the lid 107 may include one or more indicators, e.g., LED indicators, indicating the status of the battery or battery pack, the status of one or more health related parameters, a cloud connection, the uploading of measurement data or new measurement data, errors, calibration of the sensors, and the like.

Although not shown, the seat 101 and/or the lid 107 may include a user identification detecting module, e.g., a RFID detection module, which may detect a specific user based on the specific user identification, e.g., user specific RFID tags. Accordingly, the measurement data may be assigned or be regarded to a specific user of the smart toilet seat assembly 100, such as in multi-user scenarios.

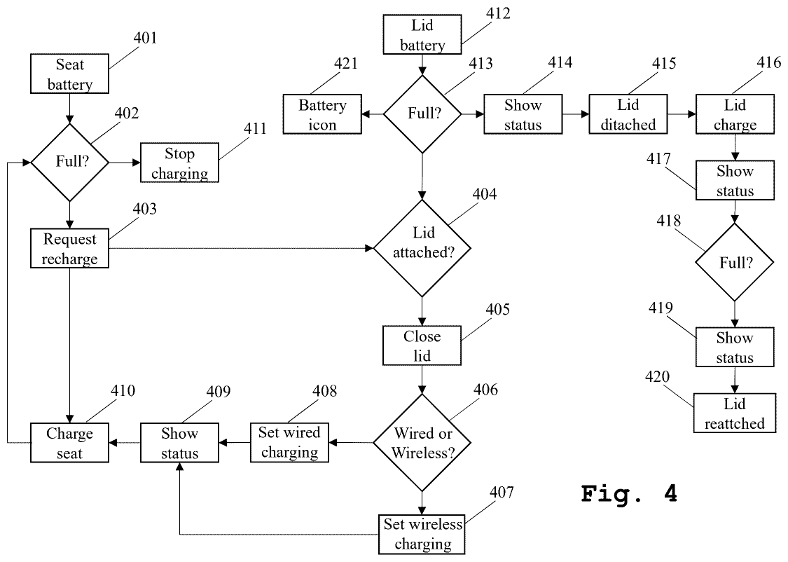

In FIG. 4, an example charging scheme of the smart toilet seat assembly 100 is illustrated. For example, during the charging step 401 of the battery 309 of the seat 101, a check 402 may be performed to obtain the charge status of the battery 309. If the battery 309 of the seat should be charged (e.g., requires charging), a recharge request 403 may be indicated, e.g., via one or more LED indicators.

Upon receiving the recharge request 403, a further check 404 may be performed to provide that the lid 107 is attached to the seat 101. If the lid 107 is attached to the seat 101, the lid 107 may be closed in the next step 405, e.g., automatic closure of the lid 107, to allow charge transfer from the battery pack 304 of the lid 107 to the battery 309 of the seat 101.

In this regard, a further check 406 may be performed to decide the mode of charge transfer. In the case of wireless charging 407, the wireless charging link between the lid 107 and the seat 101 may be set. Alternatively, the seat 101 may be directly charged 408 via a power socket or via a wired link between the lid 107 and the seat 101.

In this regard, a status 409 regarding the mode of charge transfer of the previous step 407 or 408 and/or the status of the battery 309 may be indicated, e.g., via one or more LED indicators. In a consecutive step 410, the charging or recharging of the seat 101 may be initiated. After reaching the charge capacity of the battery 309, e.g., after repeating the check 402 and if the charge capacity of the battery 309 of the seat 101 is full, the charging of the battery 309 may be stopped in the next step 411.

Alternatively, if the charge capacity of the battery 309 of the seat 101 is already full, e.g., resulted during the check 402 to obtain the charge status of the battery 309, the process flow may omit the steps 403 to 410, and may initiate the step 411 to stop charging.

For example, during the charging step 412 of the battery pack 304 of the lid 107, a check 413 may be performed to obtain the charge status of the battery pack 304 and the status 414 may be indicated, e.g., on the displays or via one or more LED indicators. Afterward, the user may detach 415 the lid 107 from the seat 101 and may connect to a power socket for charging 416, and the charging status 417 may be indicated, e.g., on the displays or via one or more LED indicators.

A further check 418 regarding the charge status of the battery pack 304 may be performed, and upon reaching the charge capacity of the battery pack 304 the charge status 419 of the battery pack 304 may be indicated, e.g., on the displays or via one or more LED indicators. Afterward, the user may reattach 420 the lid 107 to the seat 101.

Alternatively, if the charge capacity of the battery pack 304 of the lid 107 is already full, e.g., resulted during the check 413 to obtain the charge status of the battery pack 304, the process flow may omit the steps 414 to 420, and may initiate the step 421 to indicate that the battery pack 304 is at full capacity, e.g., on the displays or via one or more LED indicators.

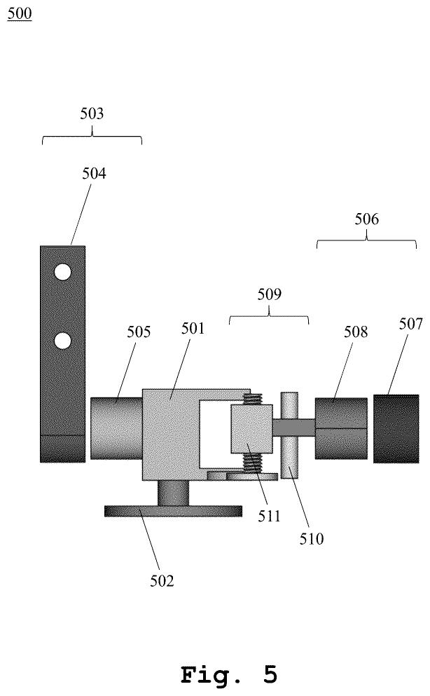

In FIG. 5, an example embodiment of the hinge 500 of the fixing arrangement is illustrated. FIG. 5 shows a left side hinge 500 of the fixing arrangement. The fixing arrangement may further include a right side hinge, and the operation of the right side hinge may be analogous to the left side hinge 500.

For example, the hinge 500 may include a hinge body that may attach the hinge 500 to the toilet bowl. The hinge body may include a main hinge body 501 and a foot 502, where the foot 502 may be detached from the toilet bowl in order to detach the hinge 500 from the toilet bowl.

For example, the hinge 500 may include a first bracket 503 coupled to the main hinge body 501. The first bracket 503 may include a lid attachment 504 that may attach the lid 107 of the smart toilet seat assembly 100 to the main hinge body 501. The lid attachment 504 may be attached to the main hinge body 501 in a rotatable manner to facilitate rotational movement of the lid 107 for opening or closing.

The first bracket 503 may further include a first damping mechanism 505 arranged between the lid attachment 504 and the main hinge body 501. In other words, the lid attachment 504 may be attached to the main hinge body 501 via the first damping mechanism 505. In this regard, the first damping mechanism 505 may provide a frictional force to control the rotational movement of the lid attachment 504 along the main hinge body 501, thereby facilitating a soft close of the lid 107.

For example, the hinge 500 may include a second bracket 506 coupled to the main hinge body 501 via a coupler 509. The second bracket 506 may include a seat attachment 507 that may attach the seat 101 of the smart toilet seat assembly 100 to the main hinge body 501 via the coupler 509. The seat attachment 507 may be attached to the main hinge body 501 via the coupler 509 in a rotatable manner to facilitate rotational movement of the seat 101.

The second bracket 506 may further include a second damping mechanism 508 arranged between the seat attachment 507 and the coupler 509. In other words, the seat attachment 507 may be attached to the coupler 509 via the second damping mechanism 508. In this regard, the second damping mechanism 508 may provide a frictional force to control the rotational movement of the seat attachment 507 along coupler 509, respectively along the main hinge body 501, thereby facilitating a soft close of the seat 101.

The coupler 509 may include a fixation rod 510 and the second bracket 506 may be coupled to the fixation rod 510 such that the second bracket 506 may be moved along a vertical axis, such as along the fixation rod 510, in order to provide a floating arrangement of the seat 101. The coupler 509 may additionally include a limiting member 511 that may control the vertical movement of the second bracket 506 along the fixation rod 510.

As such, the first bracket 503 may be directly coupled to the main hinge body 501 and the weight of the lid 107 may be transferred directly to the toilet bowl via the main hinge body 501 and the foot 502. On the other hand, the second bracket 506 may float along the main hinge body 501, such as along the fixation rod 510, in order to prevent the weight applied on the seat 101 to be transferred to the hinge 500. Accordingly, for the smart toilet seat assembly 100, the weight applied on the seat 101, e.g., the weight of the user, may be (e.g., exclusively) transferred to the load sensors 108.

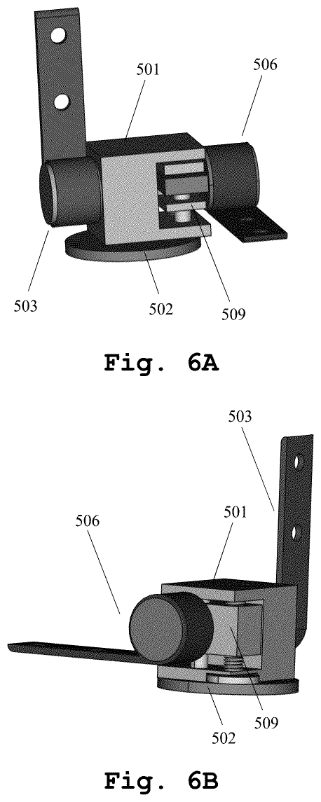

FIG. 6A shows a front view of the hinge 500 and FIG. 6B shows a back view of the hinge 500. The coupler 509 may (e.g., only) include the fixation rod 510 and the vertical motion of second bracket 506, e.g., the vertical motion of the seat 101, may be controlled by providing a fixed gap in the first damping mechanism 505 of the first bracket 503, which may (e.g., automatically) fix the vertical range.

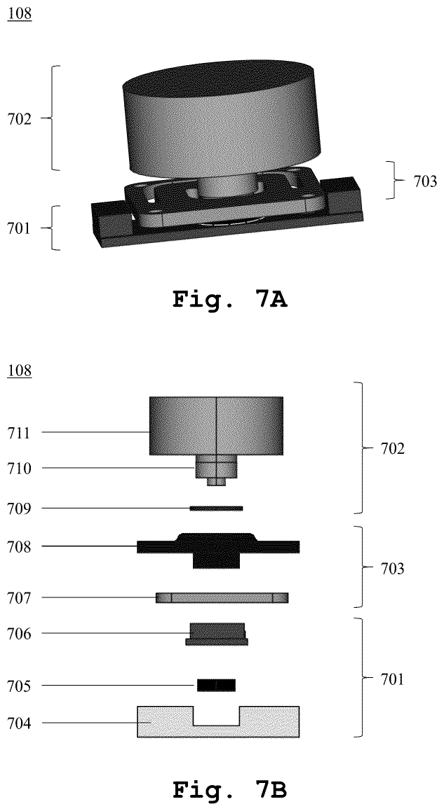

In FIG. 7A and FIG. 7B, an example embodiment of the load sensor 108 is illustrated. For example, the load sensor 108 may include a holding segment 701 that may attach the load sensor 108 to the seat 101 of the smart toilet seat assembly 100. The load sensor 108 may further include a foot segment 702 that may be in contact with the toilet bowl when the seat 101 is used by the user. Moreover, the load sensor 108 may include a sensor body 703 arranged between the holding segment 701 and the foot segment 702. For example, the holding segment 701 and the foot segment 702 may (e.g., completely) cover the sensor body 703.

FIG. 7B shows an exploded view of the load sensor 108. For example, the holding segment 701 may include a base 704, a magnet 705, and a readout circuit 706, e.g., in the form of a PCB. For instance, the base 704 may include a groove and the magnet 705 may be arranged in the groove of the base 704.

For example, the sensor body 703 may include a load cell 707, e.g., a strain gauge load cell, operably coupled to the readout circuit 706 of the holding segment 701. For instance, the readout circuit 706 of the holding segment 701 may be connected to other readout circuits of neighboring load cells, e.g., through a further groove of the base 704 of the holding segment 701. For example, the base 704 may include a further groove corresponding to the load cell 707 such that the load cell 707 may be arranged within the base 704 of the holding segment 701.

For example, the sensor body 703 may further include a cover 708 that may (e.g., fully) cover the side of the sensor body 703 opposite to the base 704 of the holding segment 701.

The foot segment 702 may include a foot base 711 and a crest 710 protruding from the foot base 711. For example, the foot base 711 may have a larger diameter than the diameter of the crest 710 in order to support the seat 101 on the toilet bowl.

In this regard, each of the cover 708, the load cell 707, and the readout circuit 706 may include a central hole, approximately of the diameter of the crest 710 such that the crest 710 may pass through the cover 708, the load cell 707, and the readout circuit 706, and may make a magnetic contact with the magnet 705. For example, a rubber ring 709, e.g., as an additional or optional component of the foot segment 702, may be arranged along the diameter of the crest 710 to facilitate an elastic contact between the foot base 711 and the cover 708 as well as to prevent dirt ingress.

As such, the magnet 705 may guide the crest 710, thereby guiding the foot segment 702, and may hold the foot segment 702 appropriately, such as during the assembly of the load sensor 108.

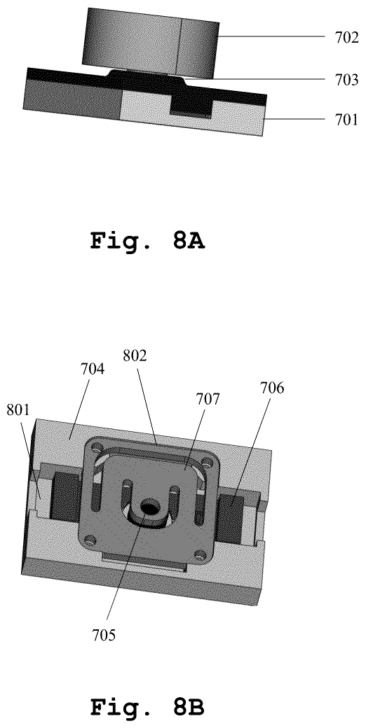

FIG. 8A shows a front view of the load sensor 108 and FIG. 8B shows a bottom inside view of the load sensor 108. FIG. 8B shows the load cell 707 inside of the base 704 of the holding segment 701. It may be seen that the groove 801 for the readout circuit 706 or the PCB may allow for a sideway extension of the electrical connection between neighboring load sensors, such as between neighboring readout circuits. It may further be seen that the groove 802 may allow the proper positioning of the load cell 707 inside of the base 704 of the holding segment 701.

In the description as well as in the claims, the word “comprising” may not exclude other elements or steps and the indefinite article “a” or “an” may not exclude a plurality. A single element or other unit may fulfill the functions of several entities or items recited in the claims.

It may be understood that the term “and/or” used in the specification and the appended claims of this application refers to any combination and (e.g., all) possible combinations of one or more associated listed items, and includes these combinations.

It may also be understood that the word “connected” or “coupled” implies that the elements may be directly connected together or may be coupled through one or more intervening elements. Moreover, the disclosure with regard to any of the aspects is also relevant with regard to the other aspects of the disclosure.

Although the disclosure has been illustrated and described with respect to one or more implementations, equivalent alterations, and modifications may occur to those skilled in the art upon the reading and understanding of this specification and the drawings. In addition, while a (e.g., particular) feature of the disclosure may have been disclosed with respect to (e.g., only) one of several implementations, such feature may be combined with one or more other features of the other implementations as may be desired for any application.

While some embodiments have been illustrated and described in detail in the appended drawings and the foregoing description, such illustration and description are to be considered illustrative and not restrictive. Other variations to the disclosed embodiments may be understood and effected in practicing the claims, from a study of the drawings, the disclosure, and the appended claims. The mere fact that (e.g., certain) measures or features are recited in mutually different dependent claims may not indicate that a combination of these measures or features may not be used. Any reference signs in the claims may not be construed as limiting the scope.

What is claimed is:

1. A smart toilet seat assembly for a toilet bowl for measuring physiological parameters of a user, the smart toilet seat assembly comprises:
a seat comprising a plurality of physiological sensors, each of the plurality of physiological sensors configured to generate at least one physiological parameter measurement data of the user, and a plurality of load sensors configured to support the seat on the toilet bowl when used by the user and further to generate a weight measurement data of the user; and
a fixing arrangement configured to attach a lid to the seat and further to attach the smart toilet seat assembly to the toilet bowl, wherein the fixing arrangement is configured to transfer a weight of the lid to the toilet bowl and further to transfer a weight applied on the seat to the plurality of load sensors.

2. The smart toilet seat assembly of claim 1,
wherein the plurality of physiological sensors are arranged on a top surface of the seat in physical contact with the user.

3. The smart toilet seat assembly of claim 1,
wherein at least one sensor of the plurality of physiological sensors is configured to be in contact with the user.

4. The smart toilet seat assembly of claim 1,
wherein the plurality of load sensors are arranged on a bottom surface of the seat in contact with the toilet bowl.

5. The smart toilet seat assembly of claim 1,
wherein the plurality of physiological sensors comprise at least one electrocardiogram sensor, at least one bio-impedance sensor, at least one photoplethysmography sensor, and at least one temperature sensor.

6. The smart toilet seat assembly of claim 1,
wherein the at least one physiological parameter measurement data comprises an electrocardiogram measurement data, a bio-impedance measurement data, a photoplethysmography measurement data, or a temperature measurement data of the user.

7. The smart toilet seat assembly of claim 1,
wherein the seat comprises a switching arrangement configured to switch on the plurality of physiological sensors or the plurality of load sensors based on a presence of the user, and
wherein the switching arrangement is configured to switch off the plurality of physiological sensors or the plurality of load sensors based on an absence of the user.

8. The smart toilet seat assembly of claim 1,
wherein the lid comprises a receiving unit configured to receive the at least one physiological parameter measurement data from the plurality of physiological sensors.

9. The smart toilet seat assembly of claim 1,
wherein the lid comprises a receiving unit configured to receive the weight measurement data from the plurality of load sensors.

10. The smart toilet seat assembly of claim 1,
wherein the lid comprises a processing unit configured to process the at least one physiological parameter measurement data or the weight measurement data and further to extract one or more physiological parameters of the user from the at least one physiological parameter measurement data or the weight measurement data.

11. The smart toilet seat assembly of claim 10,
wherein the lid comprises a display unit configured to display a notification corresponding to the extracted one or more physiological parameters of the user.

12. The smart toilet seat assembly of claim 11,
wherein the lid comprises a transmitting unit configured to transmit the one or more physiological parameters of the user to a remote device or to a cloud storage.

13. The smart toilet seat assembly of claim 12, wherein transmission of the one or more physiological parameters is performed wirelessly.

14. The smart toilet seat assembly of claim 1,
wherein the seat comprises a supply unit configured to provide an electrical supply to the plurality of physiological sensors or the plurality of load sensors.

15. The smart toilet seat assembly of claim 14,
wherein the lid comprises a charging unit operably coupled to the supply unit of the seat, the charging unit configured to transmit an electrical power to the supply unit to charge or recharge the supply unit.

16. A load sensor for generating a weight measurement data in a smart toilet seat assembly,
wherein the smart toilet seat assembly includes
a seat comprising a plurality of physiological sensors, each of the plurality of physiological sensors configured to generate at least one physiological parameter measurement data of a user, and at least one load sensor configured to support the seat on a toilet bowl when used by the user and further to generate a weight measurement data of the user, and
a fixing arrangement configured to attach a lid to the seat and further to attach the smart toilet seat assembly to the toilet bowl, wherein the fixing arrangement is configured to transfer a weight of the lid to the toilet bowl and further to transfer a weight applied on the seat to the load sensor,
the load sensor comprises:
a holding segment configured to attach the load sensor to a seat of the smart toilet seat assembly;
a foot segment configured to be in contact with a toilet bowl when used by a user; and
a sensor body comprising a load cell, the sensor body arranged between the holding segment and the foot segment,
wherein the foot segment comprises a crest configured to protrude towards the holding segment through the sensor body, and
wherein the holding segment comprises a magnet configured to align the crest of the foot segment through the sensor body and further to attach the foot segment with the holding segment.

17. The load sensor of claim 16,
wherein the load cell is a strain gauge load cell.

18. A fixing arrangement for attaching a smart toilet seat assembly to a toilet bowl, wherein
the smart toilet seat assembly includes
a seat comprising a plurality of physiological sensors, each of the plurality of physiological sensors configured to generate at least one physiological parameter measurement data of a user, and at least one load sensor configured to support the seat on the toilet bowl when used by the user and further to generate a weight measurement data of the user, and
a fixing arrangement configured to attach a lid to the seat and further to attach the smart toilet seat assembly to the toilet bowl, wherein the fixing arrangement is configured to transfer a weight of the lid to the toilet bowl and further to transfer a weight applied on the seat to the at least one load sensor,
the fixing arrangement comprises at least one hinge comprising:
a hinge body configured to attach the at least one hinge to the toilet bowl;
a first bracket coupled to the hinge body, wherein the first bracket is configured to attach a lid of the smart toilet seat assembly to the hinge body to transfer a weight of the lid to the toilet bowl and further to rotate the lid with respect to the hinge body; and
a second bracket coupled to the hinge body via a coupler, wherein the second bracket is configured to attach a seat of the smart toilet seat assembly to the hinge body via the coupler and further to rotate the seat with respect to the hinge body,
wherein the coupler is configured to move the seat along a vertical axis based on a weight applied on the seat.

19. The fixing arrangement of claim 18,
wherein the coupler comprises a limiting member configured to control movement of the seat along the vertical axis.

20. The fixing arrangement of claim 18,
wherein the first bracket comprises a first damping mechanism configured to control rotational movement of the lid with respect to the hinge body, or
wherein the second bracket comprises a second damping mechanism configured to control rotational movement of the seat with respect to the hinge body.
